(19) United States
(12) Patent Application Publication
(10) Pub. No.: US 2025/0127655 A1
(43) Pub. Date: Apr. 24, 2025

(54) Systems, Methods, and Apparatus for Automated Feedback for Targeted Temperature Management Systems

(71) Applicant: C. R. Bard, Inc., Franklin Lakes, NJ (US)

(72) Inventors: Austin J. McKinnon, Herriman, UT (US); Jett Jousma, Litchfield Park, AZ (US)

(21) Appl. No.: 18/715,064

(22) PCT Filed: Dec. 2, 2021
(86) PCT No.: PCT/US2021/061642

§ 371 Date: May 30, 2024

(51) Int. Cl. A61F 7/00

(52) U.S. Cl. CPC A61F 7/0085; A61F 2007/0054; A61F 2007/0093; A61F 2007/0096

(57) ABSTRACT

Disclosed herein are systems, methods, and apparatuses for obtaining temperature data from a temperature sensing medical instrument. The targeted temperature management (TTM) system includes a TTM module configured to provide a TTM fluid, a thermal pad configured to receive the TTM fluid from the TTM module to facilitate thermal energy transfer between the TTM fluid and a patient, a fluid delivery line (FDL) extending between the TTM module and the thermal pad, the FDL configured to provide TTM fluid flow between the TTM module and the thermal pad, and a temperature sensing medical instrument configured to be inserted into the patient and obtain temperature data indicating a temperature of blood of the patient, wherein the temperature sensing medical instrument is further configured to communicatively couple to the TTM module and provide the TTM module with temperature feedback including the temperature of the blood of the patient.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled to a patient to affect surface-to-surface thermal energy exchange with the patient. In general, TTM systems comprise a TTM fluid control module coupled to at least one contact pad via a fluid deliver line. One such TTM system is disclosed in U.S. Pat. No. 6,645,232, titled “Patient Temperature Control System with Fluid Pressure Maintenance” filed Oct. 11, 2001, and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain intimate pad-to-patient contact is of importance to fully realizing medical efficacies with TTM systems.

Further, it is integral to the efficacy of the TTM and to the health and wellbeing of the patient to accurately monitor the patient temperature. As the TTM fluid is cooled/heated and circulated through the one or more thermal contact pads, the patient temperature may change rapidly. Specifically, as the TTM fluid circulates through the thermal contact pads, the temperature of the blood flowing through the patient body at the corresponding regions is altered accordingly and the blood subsequently circulates elsewhere in the body. For instance, blood that has been cooled as a result of the TTM procedure may circulate to the brain. Accurately determining the temperature of the brain is critical in TTM procedures directed at cooling a stroke patient to gain some degree of neuroprotection, for example. It has been found that measuring the patient temperature at the patient's core, or through a thermometer attached to an appendage (e.g., a finger, arm, leg), does not accurately measure the temperature of the brain. Thus, what is needed, and what is disclosed herein, are systems, methods and apparatus for measuring blood temperature of the brain at a location immediately adjacent the brain and utilizing such measurements as feedback to the TTM system.

### SUMMARY OF THE INVENTION

Briefly summarized, disclosed herein is a targeted temperature management (TTM) system comprising a TTM module configured to provide a TTM fluid, a thermal pad configured to receive the TTM fluid from the TTM module to facilitate thermal energy transfer between the TTM fluid and a patient, a fluid delivery line (FDL) extending between the TTM module and the thermal pad, the FDL configured to provide TTM fluid flow between the TTM module and the thermal pad, and a temperature sensing medical instrument configured to be inserted into the patient and obtain temperature data indicating a temperature of blood of the patient, wherein the temperature sensing medical instrument is further configured to communicatively couple to the TTM module and provide the TTM module with temperature feedback including the temperature of the blood of the patient.

In some embodiments, the temperature sensing medical instrument includes a thermistor. In some embodiments, the temperature sensing medical instrument includes a housing and an elongate portion extending distally from the housing, wherein the thermistor is disposed along or at a distal tip of the thermistor. In some embodiments, the housing is configured to receive a catheter such that the elongate portion extends through the catheter. In some embodiments, the thermistor is configured to be disposed within an internal jugular (IJ) vein of the patient upon insertion, and wherein the temperature data obtained when the thermistor is within the IJ vein is indicative of a temperature of a brain of the patient.

In some embodiments, communicative coupling is established through establishing a connection between the TTM module and the temperature sensing medical instrument via one or more electrical wires. In some embodiments, the communicative coupling is established through establishing a wireless connection between the TTM module and the temperature sensing medical instrument in accordance with a short-range wireless technology standard. In some embodiments, the temperature sensing medical instrument includes a processor, an analog-to-digital converter, and a battery.

In some embodiments, the TTM module includes a processor and a non-transitory, computer-readable medium having stored thereon logic that, when executed by the processor, is configured to cause performance of operations including receiving the temperature data, analyzing an efficacy a TTM procedure being performed by TTM system based on the temperature data, wherein the TTM procedure includes providing the TTM fluid, and altering the TTM procedure based on the analyzing.

Also disclosed herein is a temperature sensing medical instrument configured to be inserted into a patient and obtain temperature data indicating a temperature of blood of the patient, the temperature sensing medical instrument comprising a housing configured to remain external the patient, an elongate portion extending distally from the housing and configured to be inserted into the patient, and a thermistor disposed along or at a distal tip of the thermistor, wherein the temperature sensing medical instrument is configured to communicatively couple with a targeted temperature management (TTM) module of a TTM system and provide the TTM module with the temperature data.

In some embodiments, the housing is configured to receive a catheter such that the elongate portion extends through the catheter. In some embodiments, the thermistor is configured to be disposed within an internal jugular (IJ) vein of the patient upon insertion, and wherein the temperature data obtained when the thermistor is within the IJ vein is indicative of a temperature of a brain of the patient. In some embodiments, a communicative coupling is established through a physical connection between the TTM module and the temperature sensing medical instrument via one or more electrical wires. In some embodiments, a communicative coupling is established through a wireless connection between the TTM module and the temperature sensing medical instrument in accordance with a short-range wireless technology standard. In some embodiments, temperature sensing medical instrument further comprises a processor, an analog-to-digital converter, and a battery.

Also disclosed herein is a method of using a targeted temperature management (TTM) system, comprising providing a TTM system including a TTM module configured to provide a TTM fluid, a thermal pad configured to receive the TTM fluid from the TTM module to facilitate thermal energy transfer between the TTM fluid and a patient, a fluid delivery line (FDL) extending between the TTM module and the thermal pad, the FDL configured to provide TTM fluid flow between the TTM module and the thermal pad, and a temperature sensing medical instrument configured to be inserted into the patient and obtain temperature data indicating a temperature of blood of the patient, wherein the temperature sensing medical instrument is further configured to communicatively couple to the TTM module and provide the TTM module with temperature feedback including the temperature of the blood of the patient. The method further includes applying the thermal pad to the patient, inserting the temperature sensing medical instrument into the patient, delivering TTM fluid from the TTM module to the thermal pad, receiving, by the TTM module, the temperature data from the temperature sensing medical instrument, and analyzing an efficacy a TTM procedure being performed by TTM system based on the temperature data, wherein the TTM procedure includes providing the TTM fluid.

In some embodiments, the method further comprises altering the TTM procedure based on the analyzing. In some embodiments, the temperature sensing medical instrument includes a thermistor. In some embodiments, the temperature sensing medical instrument includes a housing and an elongate portion extending distally from the housing, wherein the thermistor is disposed along or at a distal tip of the thermistor.

In some embodiments, the housing is configured to receive a catheter such that the elongate portion extends through the catheter. In some embodiments, the thermistor is configured to be disposed within an internal jugular (IJ) vein of the patient upon insertion, and wherein the temperature data obtained when the thermistor is within the IJ vein is indicative of a temperature of a brain of the patient. In some embodiments, a communicative coupling is established through establishing a connection between the TTM module and the temperature sensing medical instrument via one or more electrical wires. In some embodiments, a communicative coupling is established through establishing a wireless connection between the TTM module and the temperature sensing medical instrument in accordance with a short-range wireless technology standard.

In some embodiments, the temperature sensing medical instrument includes a processor, an analog-to-digital converter, and a battery. In some embodiments, the TTM module includes a processor and a non-transitory, computer-readable medium having stored thereon logic that, when executed by the processor, is configured to cause performance of operations including receiving the temperature data, analyzing an efficacy a TTM procedure being performed by TTM system based on the temperature data, wherein the TTM procedure includes providing the TTM fluid, and altering the TTM procedure based on the analyzing.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a patient and a targeted temperature management (TTM) system for cooling or warming the patient, in accordance with some embodiments.

FIG. 2 illustrates a hydraulic schematic of the TTM system of FIG. 1, in accordance with some embodiments.

FIG. 3 illustrates a block diagram depicting various elements of a console of the TTM module of FIG. 1, in accordance with some embodiments.

FIG. 4A is a top view of a portion of the thermal contact pad of FIG. 1, in accordance with some embodiments.

FIG. 4B is a cross-sectional side view of the portion the thermal contact pad of FIG. 4A, in accordance with some embodiments.

FIG. 5A illustrates a first embodiment of a temperature sensing medical instrument configured to communicatively coupled with the TTM system of FIG. 1, in accordance with some embodiments.

FIG. 5B illustrates the patient and the TTM system of FIG. 1 communicatively coupled to the temperature sensing medical instrument of FIG. 5A, in accordance with some embodiments.

FIG. 6A illustrates a second embodiment of a temperature sensing medical instrument configured to communicatively coupled with the TTM system of FIG. 1, in accordance with some embodiments.

FIG. 6B illustrates the patient and the TTM system of FIG. 1 communicatively coupled to the temperature sensing medical instrument of FIG. 6A, in accordance with some embodiments.

FIG. 7 is a flowchart of a method for using the TTM system of FIG. 1 to receive temperature data from the temperature sensing medical instrument of either FIG. 5A or FIG. 6A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Unless defined otherwise, all technical and scientific terms used herein have the same meaning as commonly understood by those of ordinary skill in the art.

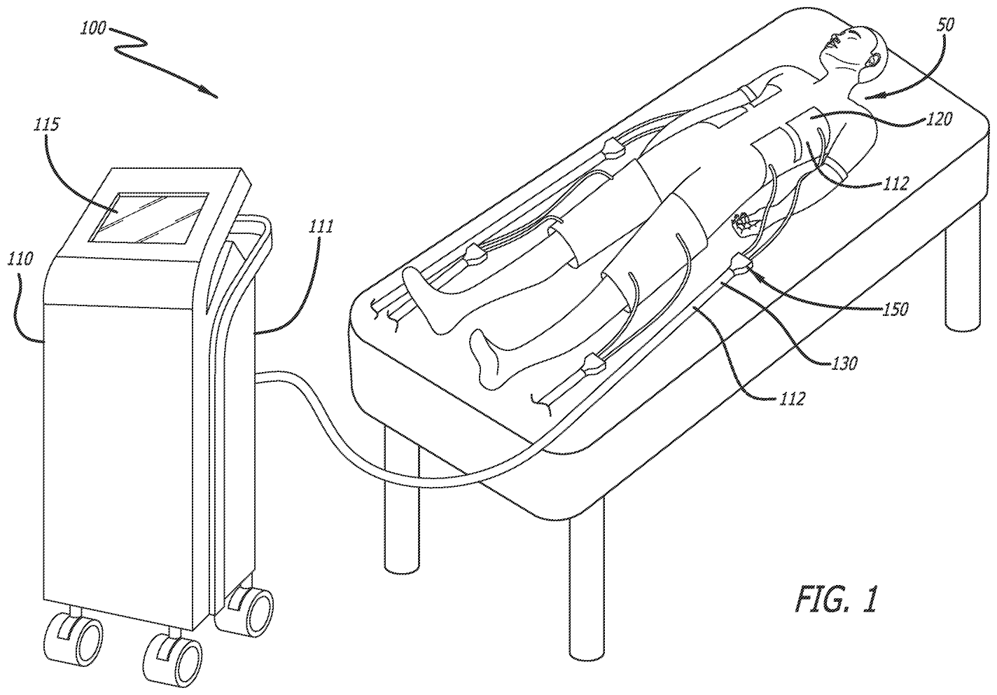

FIG. 1 illustrates a targeted temperature management (TTM) system 100 connected to a patient 50 for administering targeted temperature management therapy to the patient 50 which may include a cooling and/or warming of the patient 50, in accordance with some embodiments. The TTM system 100 comprises a TTM module 110 including a graphical user interface (GUI) 115 enclosed within a module housing 111. The TTM system 100 includes a fluid deliver line (FDL) 130 extending from the TTM module 110 to a thermal contact pad (pad) 120 to provide for flow of TTM fluid 112 between the TTM module 110 and the pad 120. The FDL includes two conduits to facilitate delivery flow of TTM fluid 112 from the TTM module 110 to the pad 120 and return flow TTM fluid 112 from the pad 120 to the TTM module 110. In some embodiments, the two conduits may be attached to each other along a portion of a length of the FDL.

The TTM system 100 may include 1, 2, 3, 4 or more pads 120 and the TTM system 100 may include 1, 2, 3, 4 or more fluid delivery lines 130. In use, the TTM module 110 prepares the TTM fluid 112 for delivery to the pad 120 by heating or cooling the TTM fluid 112 to a defined temperature in accordance with a prescribed TTM therapy. The TTM module 110 circulates the TTM fluid 112 along a TTM fluid flow path including within the pad 120 to facilitate thermal energy exchange with the patient 50. During the TTM therapy, the TTM module 110 may continually control the temperature of the TTM fluid 112 toward a target TTM temperature.

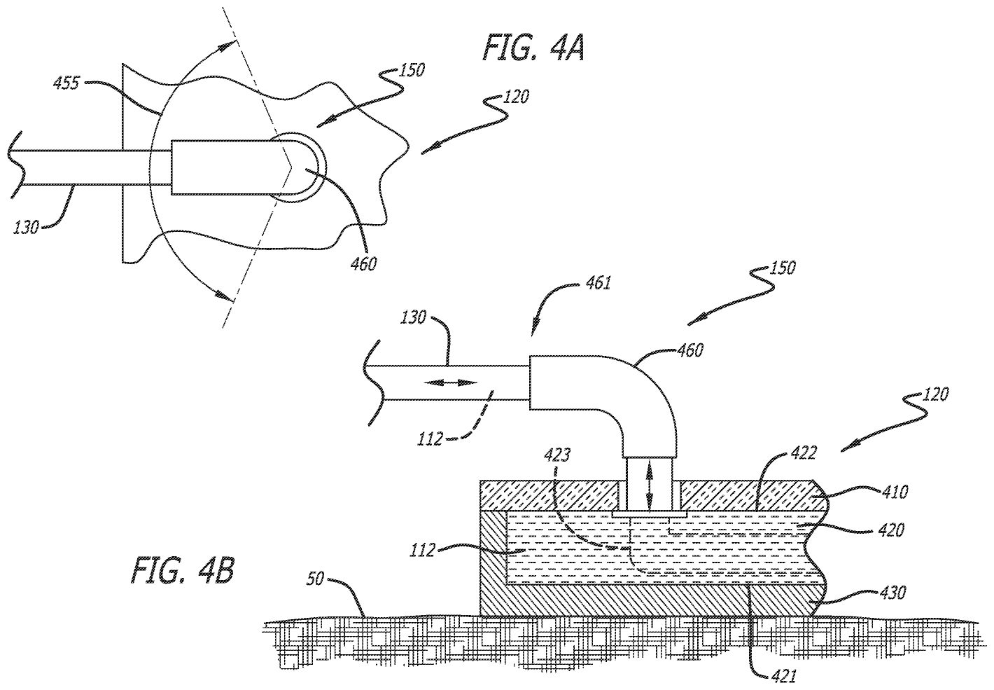

The TTM system 100 may include a connector system 150 to couple the FDL 130 to the pad 120. In some embodiments, the connector system 150 may couple a single fluid conduit of the FDL to the pad 120. Hence, the connection between the FDL 130 and the pad 120 may comprise more than one connector system 150 to couple more than one fluid conduit to the pad 120. The connector system 150 is further described below in FIGS. 4A and 4B.

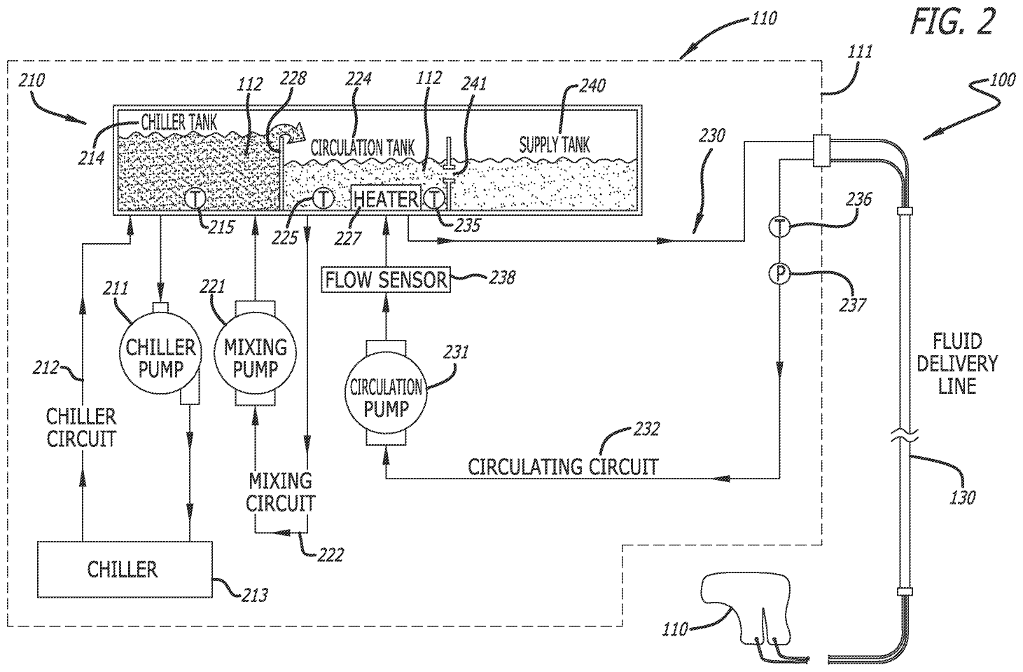

FIG. 2 illustrates a hydraulic schematic of the TTM system 100. The FDL 130 and the pad 120 are disposed external to the housing 111 of the TTM module 110. The TTM module includes various fluid sensors and fluid control devices to prepare and circulate the TTM fluid 112. The fluid subsystems of the TTM module may include a temperature control subsystem 210 and a circulation subsystem 230.

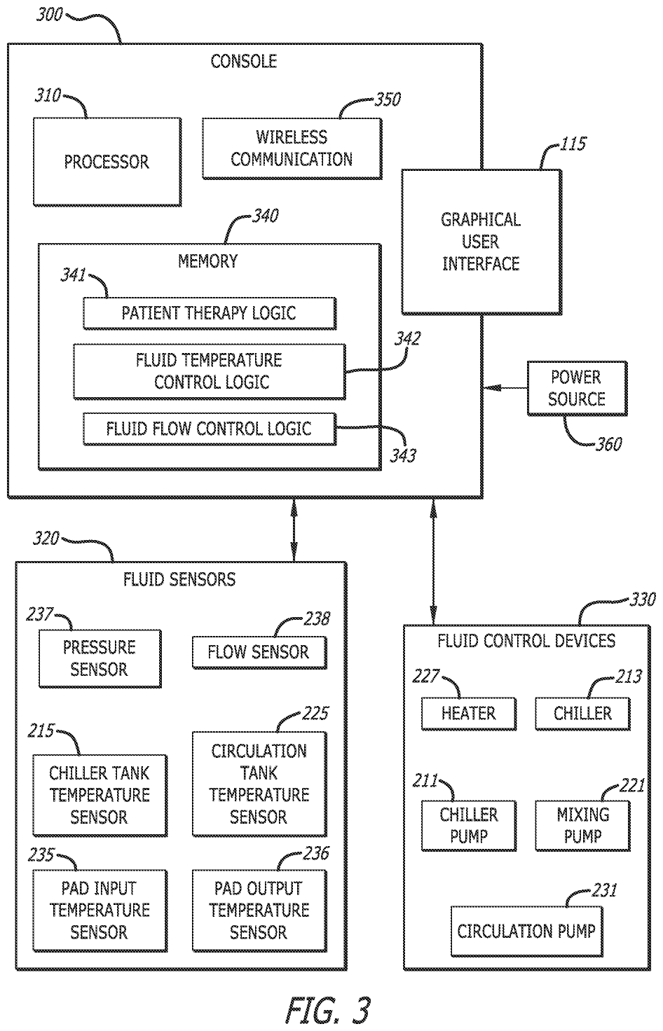

The temperature control subsystem 210 may include a chiller pump 211 to pump (recirculate) TTM fluid 112 through a chiller circuit 212 that includes a chiller 213 and a chiller tank 214. A temperature sensor 215 within the chiller tank 214 is configured to measure a temperature of the TTM fluid 112 within the chiller tank 214. The chiller 213 may be controlled by a temperature control logic (see FIG. 3) as further described below to establish a desired temperature of the TTM fluid 112 within chiller tank 214. In some instances, the temperature of the TTM fluid 112 within the chiller tank 214 may be less than the target temperature for the TTM therapy.

The temperature control subsystem 210 may further include a mixing pump 221 to pump TTM fluid 112 through a mixing circuit 222 that includes the chiller tank 214, a circulation tank 224, and a dam 228 disposed between the chiller tank 214 and circulation tank 224. The TTM fluid 112, when pumped by the mixing pump 221, enters the chiller tank 214 and mixes with the TTM fluid 112 within the chiller tank 214. The mixed TTM fluid 112 within the chiller tank 214 flows over the dam 228 and into the circulation tank 224. In other words, the mixing circuit 222 mixes the TTM fluid 112 within chiller tank 214 with the TTM fluid 112 within circulation tank 224 to cool the TTM fluid 112 within the circulation tank 224. A temperature sensor 225 within the circulation tank 224 measures the temperature of the TTM fluid 112 within the circulation tank 224. The temperature control logic may control the mixing pump 221 in accordance with temperature data from the temperature sensor 225 within the circulation tank 224.

The circulation tank 224 includes a heater 227 to increase to the temperature of the TTM fluid 112 within the circulation tank 224, and the heater 227 may be controlled by the temperature control logic. In summary, the temperature control logic when executed by the processor (see FIG. 3) May 1) receive temperature data from the temperature sensor 215 within the chiller tank and the temperature sensor 225 within the circulation tank 224 and 2) control the operation of the chiller 213, the chiller pump 211, the heater 227, and mixing pump 222 to establish and maintain the temperature of the TTM fluid 112 within the circulation tank 224 at the target temperature for the TTM therapy.

The circulation subsystem 230 comprises a circulation pump 213 to pull TTM fluid 112 from the circulation tank 224 and through a circulating circuit 232 that includes the fluid delivery line 130 and the pad 120 located upstream of the circulation pump 213. The circulating circuit 232 also includes a pressure sensor 237 to represent a pressure of the TTM fluid 112 within the pad 120. The circulating circuit 232 includes a temperature sensor 235 within the circulation tank 224 to represent the temperature of the TTM fluid 112 entering the pad 120 and a temperature sensor 236 to represent the temperature of the TTM fluid exiting the pad 120. A flow meter 238 is disposed downstream of the circulation pump 213 to measure the flow rate of TTM fluid 112 through the circulating circuit 232 before the TTM fluid 112 re-enters that the circulation tank 224.

In use, the circulation tank 224, which may be vented to atmosphere, is located below (i.e., at a lower elevation than) the pad 120 so that a pressure within the pad 120 is less than atmospheric pressure (i.e., negative) when TTM fluid flow through the circulating circuit 232 is stopped. The pad 120 is also placed upstream of the circulation pump 231 to further establish a negative pressure within the pad 120 when the circulation pump 213 is operating. The fluid flow control logic (see FIG. 3) may control the operation of the circulation pump 213 to establish and maintain a desired negative pressure within the pad 120. A supply tank 240 provides TTM fluid 112 to the circulation tank 224 via a port 241 to maintain a defined volume of TTM fluid 112 within the circulation tank 224.

FIG. 3 illustrates a block diagram depicting various elements of the TTM module 110 of FIG. 1, in accordance with some embodiments. The TTM module 110 includes a console 300 including a processor 310 and memory 340 including non-transitory, computer-readable medium. Logic modules stored in the memory 340 include patient therapy logic 341, fluid temperature control logic 342, and fluid flow control logic 343. The logic modules when executed by the processor 310 define the operations and functionality of the TTM Module 110.

Illustrated in the block diagram of FIG. 3 are fluid sensors 320 as described above in relation to FIG. 2. Each of the fluid sensors 320 are coupled to the console 300 so that data from the fluid sensors 320 may be utilized in the performance of TTM module operations. Fluid control devices 330 are also illustrated in FIG. 3 as coupled to the console 300. As such, logic modules may control the operation of the fluid control devices 330 as further described below.

The patient therapy logic 341 may receive input from the clinician via the GUI 115 to establish operating parameters in accordance with a prescribed TTM therapy. Operating parameters may include a target temperature for the TTM fluid 112 and/or a thermal energy exchange rate which may comprise a time-based target temperature profile. In some embodiments, the fluid temperature control logic 342 may define other fluid temperatures of the TTM fluid 112 within the TTM module 110, such a target temperature for the TTM fluid 112 within the chiller tank 214, for example.

The fluid temperature control logic 342 may perform operations to establish and maintain a temperature of the TTM fluid 112 delivered to the pad 120 in accordance with the predefined target temperature. One temperature control operation may include chilling the TTM fluid 112 within the chiller tank 214. The fluid temperature control logic 342 may utilize temperature data from the chiller tank temperature sensor 215 to control the operation of the chiller 213 to establish and maintain a temperature of the TTM fluid 112 within the chiller tank 214.

Another temperature control operation may include cooling the TTM fluid 112 within the circulation tank 224. The fluid temperature control logic 342 may utilize temperature data from the circulation tank temperature sensor 225 to control the operation of the mixing pump 221 to decrease the temperature of the TTM fluid 112 within the circulation tank 224 by mixing TTM fluid 112 from the chiller tank 214 with TTM fluid 112 within circulation tank 224.

Still another temperature control operation may include warming the TTM fluid 112 within the circulation tank 224. The fluid temperature control logic 342 may utilize temperature data from the circulation tank temperature sensor 225 to control the operation of the heater 227 to increase the temperature of the TTM fluid 112 within the circulation tank 224.

The fluid flow control logic 343 may control the operation of the circulation pump 231. As a thermal energy exchange rate is at least partially defined by the flow rate of the TTM fluid 112 through the pad 120, the fluid flow control logic 343 may, in some embodiments, control the operation of the circulation pump 231 in accordance with a defined thermal energy exchange rate for the TTM therapy.

The console 300 may include or be couple do wireless communication module 350 to facilitate wireless communication with external devices. A power source 360 provides electrical power to the console 300.

FIG. 4A shows a top view of a portion of the thermal contact pad 120 including the connector system 150 and the FDL 130 extending away from the connector system 150, in accordance with some embodiments. As illustrated, the connector system 150 may provide for a rotatable connection between the FDL 130 and the pad 120. The rotatable connection may provide for the FDL 130 to swivel through an angle 455 ranging up to about 90 degrees, 180 degrees, or 360 degrees.

FIG. 4B shows a cross-sectional side view of an inlet or an outlet of the thermal contact pad 120 of FIG. 1 in contact with the patient 50, in accordance with some embodiments. The pad 120 may comprise multiple layers to provide multiple functions of the pad 120. A fluid containing layer 420 is fluidly coupled to the FDL 130 via the connector system 150 to facilitate circulation of the TTM fluid 112 within the fluid containing layer 420. The fluid containing layer 420 having TTM fluid 112 circulating therein defines a heat sink or a heat source for the patient 50 in accordance with a temperature of the TTM fluid 112.

The pad 120 may include a thermal conduction layer 430 disposed between the fluid containing layer 420 and the patient 50. The thermal conduction layer 430 is configured to facilitate thermal energy transfer between the fluid containing layer 420 and the patient 50. The thermal conduction layer 430 may be attached to the thermal conduction layer 430 along a bottom surface 421 of the fluid containing layer 420. The thermal conduction layer 430 may be conformable to provide for intimate contact with the patient 50. In other words, thermal conduction layer 430 may conform to a contour of the patient 50 to inhibit the presence space or air pockets between the thermal conduction layer 430 and the patient 50.

The pad 120 may include an insulation layer 410 disposed on the top side of the fluid containing layer 420. The insulation layer 410 is configured to inhibit thermal energy transfer between the fluid containing layer 420 and the environment. The insulation layer 410 may be attached to the fluid containing layer 420 along a top surface 422 of the fluid containing layer 420. In some embodiments, the insulation layer 410 may comprise one or more openings 411 extending through the insulation layer 410 to provide for coupling of the FDL 130 with the fluid containing layer 420.

The connector system 150 may include an elbow 460 to change the direction of FDL 130 extending away from the connector system 150. As shown, the direction of FDL 130 is shifted from a direction perpendicular to the pad 120 to a direction that is substantially parallel to the pad 120. The elbow 460 also establishes an orientation of a distal portion 461 of the FDL 130 to be substantially parallel to the pad 120 and/or the fluid containing layer 420.

In some embodiments, the opening 411 illustrates an inlet port to which the FDL 130 couples such that the TTM fluid 112 may enter into the fluid containing layer 420 and flow freely in a direction as dictated by the negative pressure within the pad 120 resulting from operation of the circulation pump 213. However, in other embodiments, the fluid containing layer 420 may include one or more internal flow paths (illustrated via dashed lines 423) such that the TTM fluid 112 may flow through the internal flow path(s) in a controlled manner in the as dictated by the negative pressure resulting from operation of the circulation pump 213. In some embodiments, e.g., in which FIG. 4B illustrates an inlet port, the TTM fluid 112 exits the pad 120, and specifically the fluid containing layer 420 via an outlet port (not shown) that may resemble the configuration of the as shown in FIG. 4B.

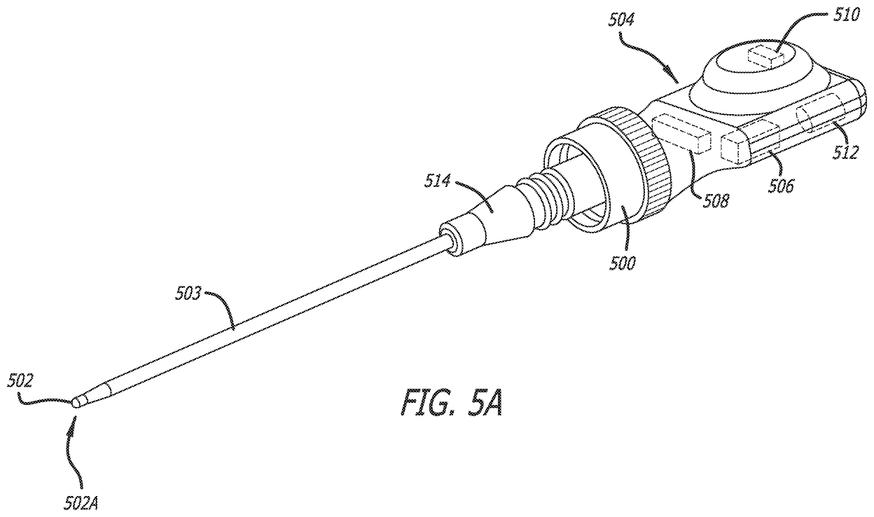

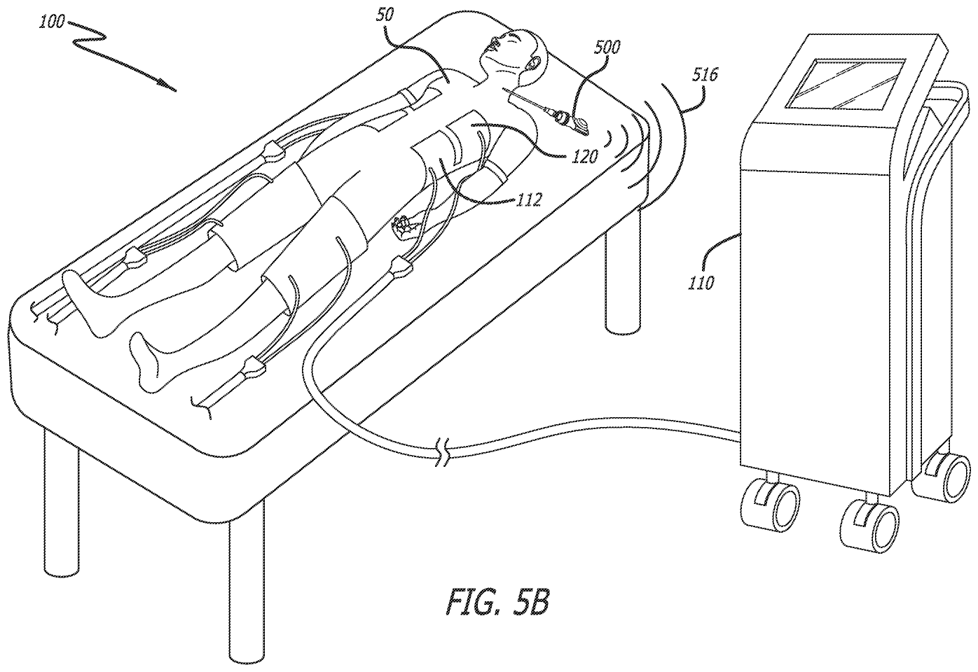

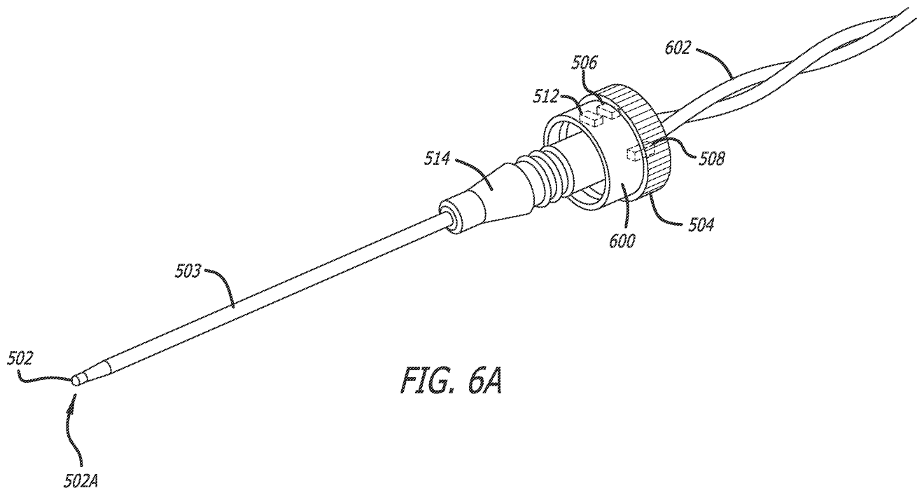

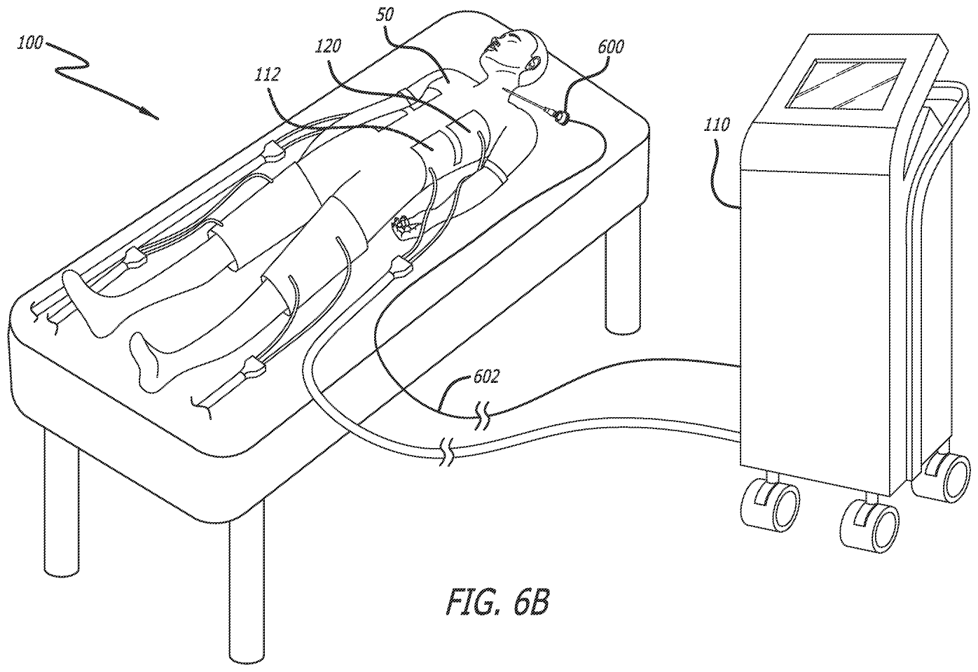

FIG. 5A illustrates a first embodiment of a temperature sensing medical instrument configured to communicatively coupled with the TTM system of FIG. 1, in accordance with some embodiments. The temperature sensing medical instrument 500 (instrument 500) includes an elongate portion 503 that extends distally into the bloodstream of a patient 50 and terminates at a distal tip 502. Further, the elongate portion 503 and the distal tip 502 are configured to extend through the catheter 514. The distal tip 502 includes a temperature sensing unit 520A that is configured to obtain temperature data of the blood of the patient 50, where the temperature data is then provided to one or more components of the instrument 500. The one or more components may be disposed in a housing 501, where the housing 501 may be configured to remain external to the patient 50 (e.g., correspond to a proximal end of the instrument 500). The one or more components may include a sensor reading module 504 (e.g., a housing), one or more processors 506, an analog-to-digital converter (ADC) 508, a communication interface (e.g., a wireless transceiver that, in some embodiments, may transmit data wirelessly in accordance with a wireless technology standard, such as in accordance with a short-range wireless technology standard, e.g., any version of the BLUETOOTH® standard) 510 and a battery 512. In some embodiments, the temperature sensing unit 520A of the distal tip 502 may be an analog temperature sensor (e.g., a low-power analog temperature sensor that may include a thermocouple or resistance temperature detector) that provides an electrical signal to the ADC 508, which is used to determine corresponding temperature data. The wireless transceiver 510 then transmits the temperature data to the TTM module 100, as seen in FIGS. 5B, 6B.

In some particular embodiments, the temperature sensing unit 520A may include a thermistor, which is a resistor whose resistance varies based on temperature. In some embodiments, the thermistor may have a length within the range of 0.012″-0.022″. In some embodiments, the thermistor need not require amplification. Additionally, the thermistor may implement chip-in-glass technology and may provide 14,0040Ω at 37° C., 25/50 Beta: 3500 nominal.

Referring now to FIG. 5B, the patient and the TTM system of FIG. 1 communicatively coupled to the temperature sensing medical instrument of FIG. 5A are shown in accordance with some embodiments. As discussed above, the elongate portion 503 (and its distal tip 502) of the instrument 500 is configured to be inserted into the patient 50. In some embodiments, the insertion site may be located on the neck of the patient 50 such that the distal tip 502 is disposed in the internal jugular (IJ) vein. The IJ vein drains blood from the brain, and thus, measuring temperature of the blood within the IJ vein advantageously provides an accurate measurement of the temperature of the brain itself. As a result, analysis of efficacy the TTM procedure may be performed with an accurate temperature measurement of the brain. As noted above, current TTM technology may utilize a patient's core temperature as patient temperature feedback to assess the efficacy of the TTM procedure or may not utilize patient temperature feedback at all. Therefore, as many TTM procedures are directed to altering the temperature of the brain (e.g., to reduce swelling and brain injuries), patient temperature feedback derived from the temperature of the blood from the brain via the IJ vein is an advantageous and novel method for assessing the efficacy of the TTM procedure. Further, as seen in FIG. 5B, the temperature data may be wirelessly transmitted (via wireless signals 516) from the instrument 500 to the module 110.

FIG. 6A illustrates a second embodiment of a temperature sensing medical instrument configured to communicatively coupled with the TTM system of FIG. 1, in accordance with some embodiments. The temperature sensing medical instrument 600 (instrument 600) includes numerous components that are included in the instrument 500 and discussed above; thus, such components will not be discussed in detail with respect to FIG. 6A. In contrast to the embodiment of FIG. 5A in which the instrument 500 in configured to transmit the temperature data wirelessly, the instrument 600 is configured to transmit the temperature data via the electrical wires 602. As shown in FIG. 6B, the patient and the TTM system of FIG. 1 are communicatively coupled to the temperature sensing medical instrument of FIG. 6A in accordance with some embodiments. In particular, the communicative coupling shown in FIG. 6B is via the electrical wires 602. In some embodiments, the wires 602 may be piped along with the fluid deliver lines (e.g., FDL 130) fluidly connecting the pads 120 and the module 110. However, in other embodiments, the wires 602 need not be piped along with the FDL 130.

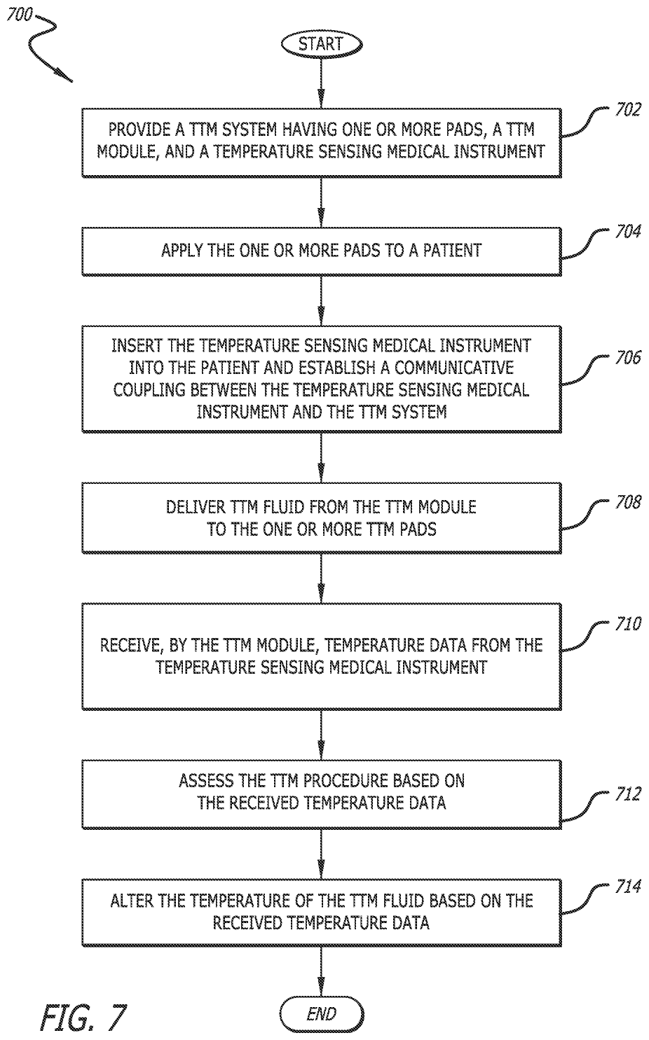

FIG. 7 is a flowchart of a method for using TTM system of FIG. 1 to receive temperature data from the temperature sensing medical instrument of either FIG. 5A or FIG. 6A, in accordance with some embodiments. Each block illustrated in FIG. 7 represents an operation included in the method 700 for using TTM system of FIG. 1 to receive temperature data from the temperature sensing medical instrument of either FIG. 5A or FIG. 6A. It should be understood that not every operation illustrated in FIG. 7 is required. The method 700 begins when a TTM system, such as the TTM system 100 of FIG. 1, is provided and includes one or more pads (e.g., the pads 120), a TTM module (e.g., the module 110) and a temperature sensing medical instrument configured to communicatively coupled with the TTM module (block 702). The temperature sensing medical instrument may be either of the instruments 500, 600. The method 700 then includes an operation of applying the one or more pads of the TTM system to a patient (block 704). One illustrative embodiment of applying the one or more pads to the patient is shown in FIG. 1, where the pads 120 are applied to various external regions of the patient body.

The method 700 continues with an operation of inserting the temperature sensing medical instrument into the patient and establishing a communicative coupling between the temperature sensing medical instrument and the TTM system (block 706). For example, inserting the temperature sensing medical instrument into the patient may include inserting an elongate portion of the temperature sensing medical instrument (see FIGS. 5A, 6A) into the neck of the patient such that the distal tip of the elongate portion is disposed within the IJ vein of the patient. Establishing a communicatively coupling may include operations of either creating a physical connection between the temperature sensing medical instrument and the TTM module via one or more electrical wires or establishing a wireless connectivity (e.g., in accordance with a wireless technology standard).

The method 700 continues with an operation of delivering TTM fluid from the TTM module to the one or more pads (block 708). The TTM module then receives temperature data from the temperature sensing medical instrument (block 710). The TTM procedure is then assessed based on the received temperature data (block 712). For example, the efficacy of the TTM procedure may be assessed by comparing the temperature of the blood of the patient within the IJ vein to an expected blood temperature. Based on a comparison (e.g., determining whether the patient blood temperature is within a predetermined tolerance of the expected blood temperature), the TTM procedure may be altered (block 714). For example, logic of the TTM module may calculate and store certain expected blood temperatures based on initial variables such as patient information (gender, age, height, weight, initial temperature), overall desired temperature, desired time for TTM procedure, etc., and subsequently calculate desire blood temperatures at sequential time segments (e.g., intervals) based thereon. Thus, the logic of the TTM module may compare the patient blood temperature obtained from the temperature sensing medical instrument against the corresponding desired blood temperature.

In some embodiments, the TTM procedure may provide TTM fluid at certain temperatures determined through the use of machine learning techniques. For instance, a trained machine learning model may be configured to provide an output indicating a current TTM fluid temperature at which the TTM fluid should be provided to the pads of the TTM system based on variables such as the initial variables referenced above, and then the subsequent variables including one or more of the patient information (replacing the initial temperature with a current temperature obtained from the temperature sensing medical instrument), overall desired temperature and, optionally, desired time for the TTM procedure. In some instances, the machine learning model may instead provide an output indicating both a current TTM fluid temperature and a remaining time for the TTM procedure. Such a machine learning model may be trained using historical TTM data (historical patient information, patient temperature data monitored during TTM procedures, corresponding desired patient temperatures, etc.). In some embodiments, the logic to train and deploy the machine learning model may be included within any of the patient therapy logic 341, the fluid temperature control logic 342, or the fluid flow control logic 343 (see FIG. 3). In some embodiments, such logic may be included in a separate logic module also stored in the memory 340.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A targeted temperature management (TTM) system, comprising:
a TTM module configured to provide a TTM fluid;
a thermal pad configured to receive the TTM fluid from the TTM module to facilitate thermal energy transfer between the TTM fluid and a patient;
a fluid delivery line (FDL) extending between the TTM module and the thermal pad, the FDL configured to provide TTM fluid flow between the TTM module and the thermal pad; and
a temperature sensing medical instrument configured to be inserted into the patient and obtain temperature data indicating a temperature of blood of the patient, wherein the temperature sensing medical instrument is further configured to communicatively couple to the TTM module and provide the TTM module with temperature feedback including the temperature of the blood of the patient.

2. The system of claim 1, wherein the temperature sensing medical instrument includes a thermistor.

3. The system of claim 2, wherein the temperature sensing medical instrument includes a housing and an elongate portion extending distally from the housing, wherein the thermistor is disposed along or at a distal tip of the elongate portion.

4. The system of claim 3, wherein the housing is configured to receive a catheter such that the elongate portion extends through the catheter.

5. The system of claim 1, wherein the thermistor is configured to be disposed within an internal jugular (IJ) vein of the patient upon insertion, and wherein the temperature data obtained when the thermistor is within the IJ vein is indicative of a temperature of a brain of the patient.

6. The system of claim 1, wherein communicative coupling is established through establishing a connection between the TTM module and the temperature sensing medical instrument via one or more electrical wires.

7. The system of claim 1, wherein the communicative coupling is established through establishing a wireless connection between the TTM module and the temperature sensing medical instrument in accordance with a short-range wireless technology standard.

8. The system of claim 1, wherein the temperature sensing medical instrument includes a processor, an analog-to-digital converter, and a battery.

9. The system of claim 1, wherein the TTM module includes a processor and a non-transitory, computer-readable medium having stored thereon logic that, when executed by the processor, is configured to cause performance of operations including:
receiving the temperature data,
analyzing an efficacy a TTM procedure being performed by TTM system based on the temperature data, wherein the TTM procedure includes providing the TTM fluid, and altering the TTM procedure based on the analyzing.

10. A temperature sensing medical instrument configured to be inserted into a patient and obtain temperature data indicating a temperature of blood of the patient, the temperature sensing medical instrument comprising:
a housing configured to remain external the patient;
an elongate portion extending distally from the housing and configured to be inserted into the patient; and
a thermistor disposed along or at a distal tip of the elongate portion, wherein the temperature sensing medical instrument is configured to communicatively couple with a targeted temperature management (TTM) module of a TTM system and provide the TTM module with the temperature data.

11. The temperature sensing medical instrument of claim 10, wherein the housing is configured to receive a catheter such that the elongate portion extends through the catheter.

12. The temperature sensing medical instrument of claim 10, wherein the thermistor is configured to be disposed within an internal jugular (IJ) vein of the patient upon insertion, and wherein the temperature data obtained when the thermistor is within the IJ vein is indicative of a temperature of a brain of the patient.

13. The temperature sensing medical instrument of claim 10, wherein communicative coupling is established through a physical connection between the TTM module and the temperature sensing medical instrument via one or more electrical wires.

14. The temperature sensing medical instrument of claim 10, wherein communicative coupling is established through a wireless connection between the TTM module and the temperature sensing medical instrument in accordance with a short-range wireless technology standard.

15. The temperature sensing medical instrument of claim 10, further comprises:
a processor;
an analog-to-digital converter; and
a battery.

16. A method of using a targeted temperature management (TTM) system, comprising:
providing a TTM system comprising:
a TTM module configured to provide a TTM fluid,
a thermal pad configured to receive the TTM fluid from the TTM module to facilitate thermal energy transfer between the TTM fluid and a patient,
a fluid delivery line (FDL) extending between the TTM module and the thermal pad, the FDL configured to provide TTM fluid flow between the TTM module and the thermal pad, and
a temperature sensing medical instrument configured to be inserted into the patient and obtain temperature data indicating a temperature of blood of the patient, wherein the temperature sensing medical instrument is further configured to communicatively couple to the TTM module and provide the TTM module with temperature feedback including the temperature of the blood of the patient;
applying the thermal pad to the patient;
inserting the temperature sensing medical instrument into the patient;
delivering TTM fluid from the TTM module to the thermal pad;
receiving, by the TTM module, the temperature data from the temperature sensing medical instrument; and
analyzing an efficacy a TTM procedure being performed by TTM system based on the temperature data, wherein the TTM procedure includes providing the TTM fluid.

17. The method of claim 16, further comprising:
altering the TTM procedure based on the analyzing.

18. The method of claim 16, wherein the temperature sensing medical instrument includes a thermistor.

19. The method of claim 18, wherein the temperature sensing medical instrument includes a housing and an elongate portion extending distally from the housing, wherein the thermistor is disposed along or at a distal tip of the elongate portion.

20. The method of claim 19, wherein the housing is configured to receive a catheter such that the elongate portion extends through the catheter.

21. The method of claim 16, wherein the thermistor is configured to be disposed within an internal jugular (IJ) vein of the patient upon insertion, and wherein the temperature data obtained when the thermistor is within the IJ vein is indicative of a temperature of a brain of the patient.

22. The method of claim 16, wherein communicative coupling is established through establishing a connection between the TTM module and the temperature sensing medical instrument via one or more electrical wires.

23. The method of claim 16, wherein communicative coupling is established through establishing a wireless connection between the TTM module and the temperature sensing medical instrument in accordance with a short-range wireless technology standard.

24. The method of claim 16, wherein the temperature sensing medical instrument includes a processor, an analog-to-digital converter, and a battery.

25. The method of claim 1, wherein the TTM module includes a processor and a non-transitory, computer-readable medium having stored thereon logic that, when executed by the processor, is configured to cause performance of operations including:
receiving the temperature data,
analyzing an efficacy a TTM procedure being performed by TTM system based on the temperature data, wherein the TTM procedure includes providing the TTM fluid, and altering the TTM procedure based on the analyzing.
